(19) United States
(12) Patent Application Publication
(10) Pub. No.: US 2024/0009389 A1
(43) Pub. Date: Jan. 11, 2024

(54) APPARATUSES AND METHODS FOR DETECTING AN EMPTY RESERVOIR IN AN INFUSION PUMP

(71) Applicant: Becton, Dickinson and Company, Franklin Lakes, NJ (US)

(72) Inventor: Scott STEWART, Pittsburgh, PA (US)

(73) Assignee: Becton, Dickinson and Company, Franklin Lakes, NJ (US)

(21) Appl. No.: 18/254,559

(22) PCT Filed: Dec. 9, 2021
(86) PCT No.: PCT/US2021/062577

§ 371 Date: May 25, 2023

Related U.S. Application Data
(60) Provisional application No. 63/125,486, filed on Dec. 15, 2020.

(51) Int. Cl. A61M 5/168

(52) U.S. Cl. CPC A61M 5/1684

(57) ABSTRACT

Devices and methods detect an empty reservoir condition in an infusion pump using pump measurements such as motor current during aspiration. An infusion pump obtains and analyzes pump measurements indicative of pressure during aspiration and determines whether pump measurements satisfy metrics corresponding to an empty reservoir condition such as pressure threshold corresponding to a pump measurement value exceeded when the reservoir is empty, a range of pump measurement values indicating a pressure above normal operating pressure of the pump, and a designated shape of a signal waveform corresponding to the pump measurements indicating a pressure above normal operating pressure of the pump. Devices and methods can be configured to disregard one or more of the pump measurements obtained during one or more portions of the duration of the aspirate operation characterized by transient increases therein from normal operation of the pumping mechanism.

### BACKGROUND

### Field

Illustrative embodiments relate generally to detecting an empty reservoir condition using pump measurement data corresponding an aspiration operation of an infusion device.

### Description of Related Art

Infusion pumps generally employ a reservoir with a known volume of fluid and known dispense stroke volume to count down doses to estimate how much fluid remains in the reservoir. Without knowing precisely the volume of fluid in the reservoir, an infusion pump can miss some doses at its end of life (e.g., end of dose countdown) such as when the dispense stroke volume was above a nominal volume.

One solution for monitoring fill level or empty state of an infusion pump reservoir is to use a dedicated sensor. Adding a sensor to the infusion pump, however, increases the complexity of the system (e.g., increases mechanical, electrical, and/or software complexity), increases system power consumption, and increases the cost of the infusion pump.

For medical devices such as a wearable medication delivery pump, where some or all of the components are disposable for ease of use and cost effectiveness, adding another component such as a reservoir status sensor and related increased cost and complexity to the medical device is undesirable. A need therefore exists for accurate detection of the empty state of a reservoir in an infusion pump without adding components and thereby increasing infusion pump complexity and cost.

### SUMMARY

The above and other problems are overcome, and additional advantages are realized, by illustrative embodiments.

In accordance with aspects of illustrative embodiments, an infusion device is provided that comprises: a pump comprising a chamber of fluid, and a pumping mechanism configured to control aspiration of a volume of fluid from a reservoir into the chamber during an aspirate operation and dispensing the fluid from the chamber during a dispense operation, and a processing device configured to analyze one or more pump measurements obtained during the aspirate operation and determine when the one or more of the pump measurements satisfies a designated metric related to an empty reservoir condition of the reservoir.

In accordance with aspects of illustrative embodiments, the pump measurements are measurements of motor current of the pump.

In accordance with aspects of illustrative embodiments, the processing device is configured to terminate operation of the pumping mechanism when the one or more of the pump measurements satisfies the designated metric.

In accordance with aspects of illustrative embodiments, the processing device is configured to analyze additional pump measurements when the one or more of the pump measurements satisfies the designated metric, and determine when the additional pump measurements satisfies the designated metric before terminate operation of the pumping mechanism. For example, the processing device can be configured to terminate operation of the pumping mechanism when the additional pump measurements satisfy the designated metric.

In accordance with aspects of illustrative embodiments, the designated metric is one or more metrics chosen from a pressure threshold corresponding to a pump measurement value exceeded when the reservoir is empty, a range of pump measurement values indicating a pressure above normal operating pressure of the pump, and a designated shape of a signal waveform corresponding to the pump measurements indicating a pressure above normal operating pressure of the pump.

In accordance with aspects of illustrative embodiments, the processing device is configured to analyze one or more of the pump measurements obtained during a selected portion of the duration of the aspirate operation.

In accordance with aspects of illustrative embodiments, the processing device is configured to disregard one or more of the pump measurements obtained during one or more portions of the duration of the aspirate operation characterized by transient increases therein from normal operation of the pumping mechanism.

In accordance with aspects of illustrative embodiments, the pump measurements are chosen from one or more of pump motor current, pump motor voltage, pump encoder count, pump motor drive count, and pump motor drive time.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims

### BRIEF DESCRIPTION OF THE DRAWINGS

The above and/or other aspects and advantages of the illustrative embodiments will be more readily appreciated from the following detailed description, taken in conjunction with the accompanying drawings, of which:

FIGS. 1 and 2 are partial, perspective views of example pump components in an example medication delivery device that operates in accordance with an occlusion detection algorithm in accordance with an illustrative embodiment of the present invention;

FIGS. 3A and 3B are perspective views of pump components of FIGS. 1 and 2 in an example medication delivery device arranged, respectively, in accordance with a ready to dispense stage of operation and a ready to aspirate stage of operation;

FIG. 3C is a perspective view of components in an example medication delivery device comprising example pump components of FIGS. 1 and 2 and associated electronic circuits on a printed circuit board;

FIG. 4A is a block diagram of components in an example medication delivery device;

FIG. 4B is a schematic diagram of a medication delivery device pump motor having a current sensor in accordance with an illustrative embodiment of the present invention;

FIG. 5 depicts pump measurement data from an example delivery device indicating motor current during dispensing and variance at different pressures;

FIGS. 6A and 6B depict, respectively, raw and filtered pump measurement data (e.g., motor current) from an example delivery device during aspirate and dispense strokes;

FIGS. 7A and 7B depict, respectively, changes in pump motor current during aspiration after the pump reservoir is empty;

FIG. 8 depicts pump measurement data from an example delivery device indicating motor current relative to aspirate strokes and an empty reservoir point; and

FIG. 9 is a flow chart of illustrative operations of an example medication delivery device performing an empty reservoir detection algorithm in accordance with an illustrative embodiment of the present invention.

Throughout the drawing figures, like reference numbers will be understood to refer to like elements, features and structures.

### DETAILED DESCRIPTION OF ILLUSTRATIVE EMBODIMENTS

Reference will now be made in detail to example embodiments of the present disclosure, which are illustrated in the accompanying drawings. The example embodiments described herein exemplify, but do not limit, the claimed invention and present disclosure by referring to the drawings.

Occlusion in a fluid pump can result from restricted flow or pathway constriction such as a pinched catheter or tissue occlusion in a fluid delivery device such as an infusion pump for medication, or from an empty medication reservoir. It is important to measure pump pressure changes from an occlusion or pump condition such as an empty reservoir for early detection to mitigate against possible fluid delivery inaccuracies resulting therefrom such as missed doses.

Some infusion pumps rely on counting down the doses to determine when their reservoirs are empty. These infusion pumps work from a known reservoir volume and a known stroke volume and count the doses until the reservoir or fluid chamber is theoretically empty. This countdown method can result in some missed doses at the end of life of the reservoir if the stroke volume is above nominal. Some of these infusion pumps require over-pumping an empty reservoir to ensure that, even at worst case conditions, all of the drug is delivered. Over-pumping causes inconvenience by increasing delivery time without providing benefit to the patient. In addition, significant power is used because aspirate strokes require excessive power when the reservoir is empty, as compared to power used for aspirate strokes when the reservoir is not empty. Another method of detecting an empty reservoir condition involves using an empty reservoir sensor, which undesirably increases infusion pump complexity, cost and possible power consumption.

Example embodiments for detecting an empty reservoir state described herein provide a technical solution to the above technical problems. In accordance with an advantageous aspect of example embodiments of the present disclosure, a pump and method for operating same are provided wherein an empty reservoir algorithm is employed to detect an empty reservoir condition using a measured pump parameter and software to control operations of a pump control device or processor based on the measured pump parameter. Knowing when a pump is empty based on a physical signal such as a measured pump parameter instead of estimation provides great benefits to pump operation including maintaining dose accuracy and reducing power consumption.

The measured pump parameter is indicative of pressure and can be, but is not limited to, any of motor current, motor voltage, encoder count, motor drive count, delivery pulse energy, motor drive time, and so on. For example, current sensing is generally considered to be a reliable method of detecting occlusions in a fluid path of a fluid delivery device due, for example, to an empty reservoir because motor current can be indirectly correlated to pump pressure. An empty reservoir causes a decrease in fluid flow in the pump, which causes increased back pressure. An increase in back pressure acting on a piston face of the pump, for example, causes an increase in torque demand required by the pump and motor to overcome this pressure. The increase in torque demand corresponds to an increase in current drawn by the pump motor, which is one way to detect downstream occlusions.

Although high positive downstream pressure can be detected based on current demand, high upstream pressure is not easily detected because high upstream pressure would theoretically aid the pump and minutely decrease current demand. However, in some positive displacement pumps, the pump first aspirates a volume of volume and then dispenses it. While it can be very difficult to detect positive upstream pressures relevant to aspirating the fluid due to decreases in current demand, it is possible to detect low pressures or events preventing upstream flow (e.g., empty reservoir) because increased current is drawn during the aspirate stroke.

In accordance with advantageous aspects of example embodiments of the present disclosure, an empty reservoir algorithm provides reliable and timely detection of an empty reservoir condition to mitigate against missed doses or otherwise inaccurate dosing that can otherwise occur at the end of life of a pump reservoir. The empty reservoir algorithm controls a fluid delivery device to obtain measurement(s) of a device parameter that is indicative of fluid pressure during intake or aspiration (e.g., an aspirate stroke), and to control a pump mechanism in the fluid delivery device to stop pump operation when the measurement(s) satisfies designated criteria corresponding to an empty reservoir condition such as a threshold (T_{EMPTY}) for the motor current or other metric such as an expected shape of the current motor data during aspiration. For example, as described below, a measured pump parameter can be monitored (e.g., motor current) during important parts of the aspirate stroke to detect empty conditions. In accordance with an underling technical principle of technical solutions described herein, pulling on an empty reservoir requires increased torque during the aspirate part of the pumping cycle. This increased torque demand increases current in a characteristic way during specific periods of the aspirate stroke. Metrics can be designated to detect in pump measurements a characteristic shape of the aspirate current draw when the pump is empty.

Example embodiments of the present disclosure are illustrated and described wherein motor current is the parameter to be measured as an indication of pressure. It is to be understood that a different pump motor parameter indicative of pressure can be measured such as, but not limited to, motor voltage, motor drive time, motor coast time, delivery pulse energy, motor drive count, motor coast count, and delta encoder count, among other parameters.

The example embodiments of an empty reservoir algorithm are particularly useful with respect to positive displacement pumps. A positive displacement pump is understood to be a type of pump that works on the principle of filling a chamber (e.g., with liquid medication from a reservoir) in one stage and then emptying the fluid from the chamber (e.g., to a delivery device such as a cannula deployed in a patient) in another stage. For example, a reciprocating plunger-type pump or a rotational metering-type pump can be used. In either case, a piston or plunger is retracted from a chamber to aspirate or draw medication into the chamber and allow the chamber to fill with a volume of medication (e.g., from a reservoir or cartridge of medication into an inlet port). The piston or plunger is then re-inserted into the chamber to dispense or discharge a volume of the medication from the chamber (e.g., via an outlet port) to a fluid pathway extending between the pump and a cannula in the patient.

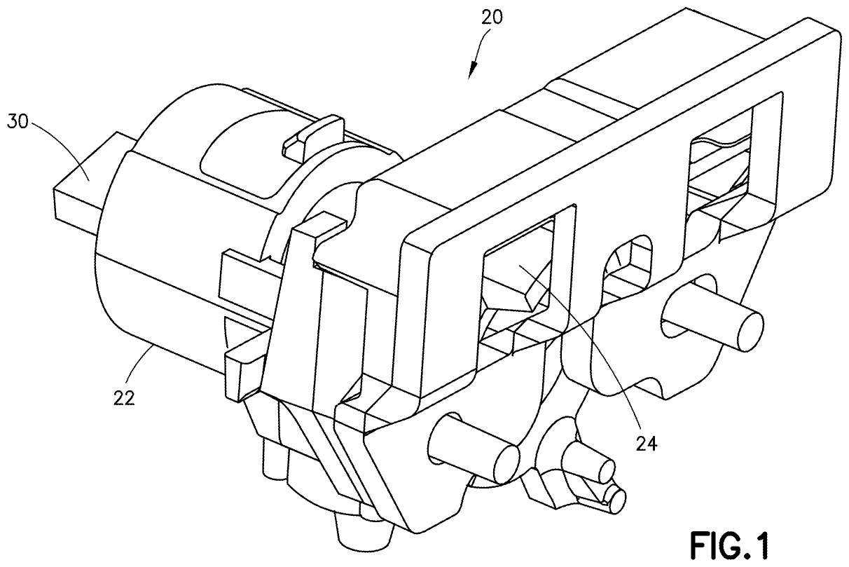

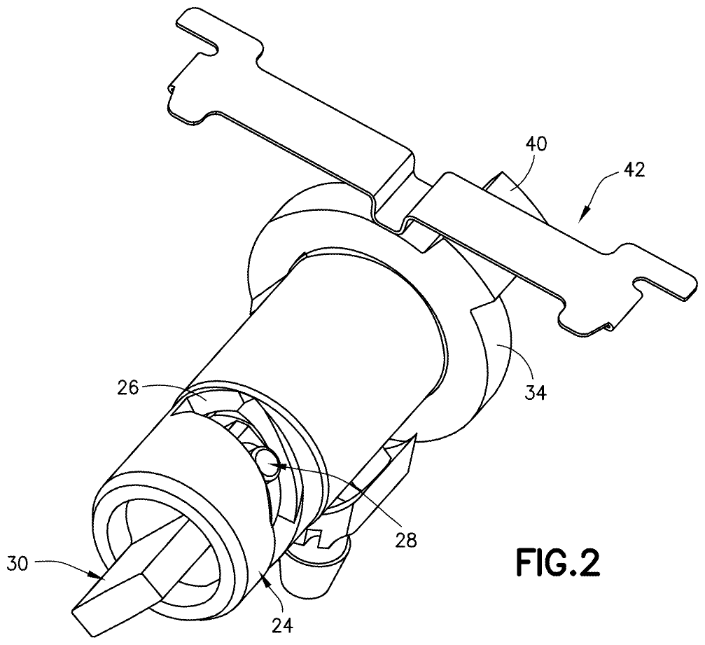

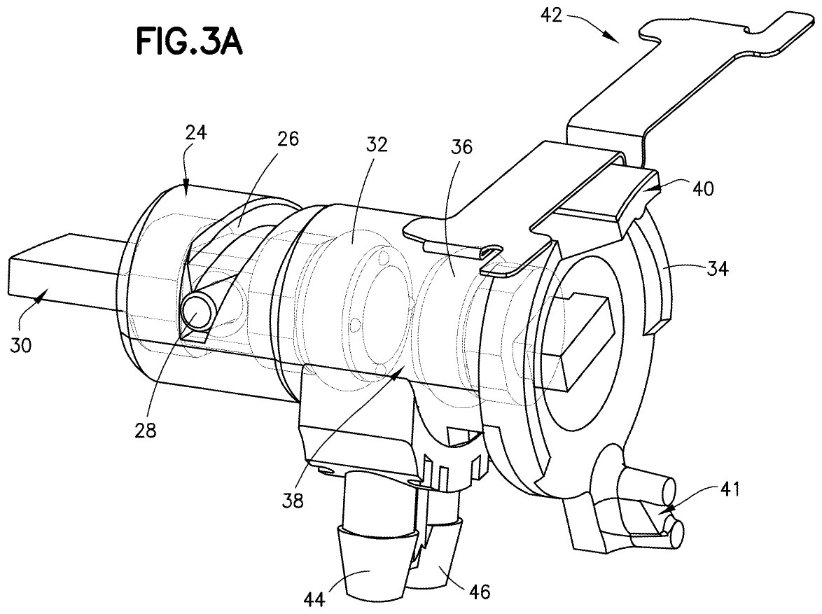

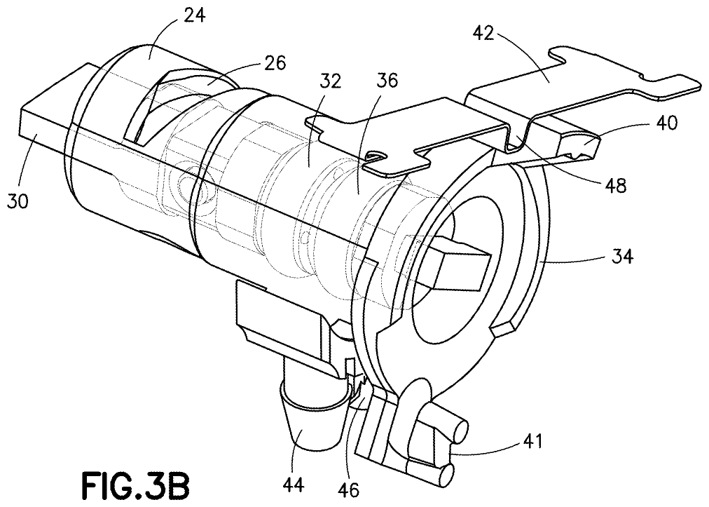

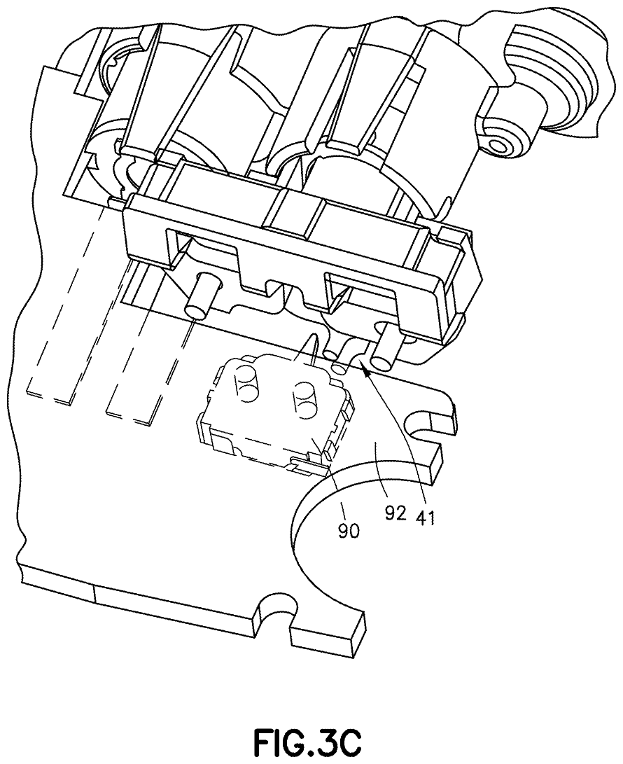

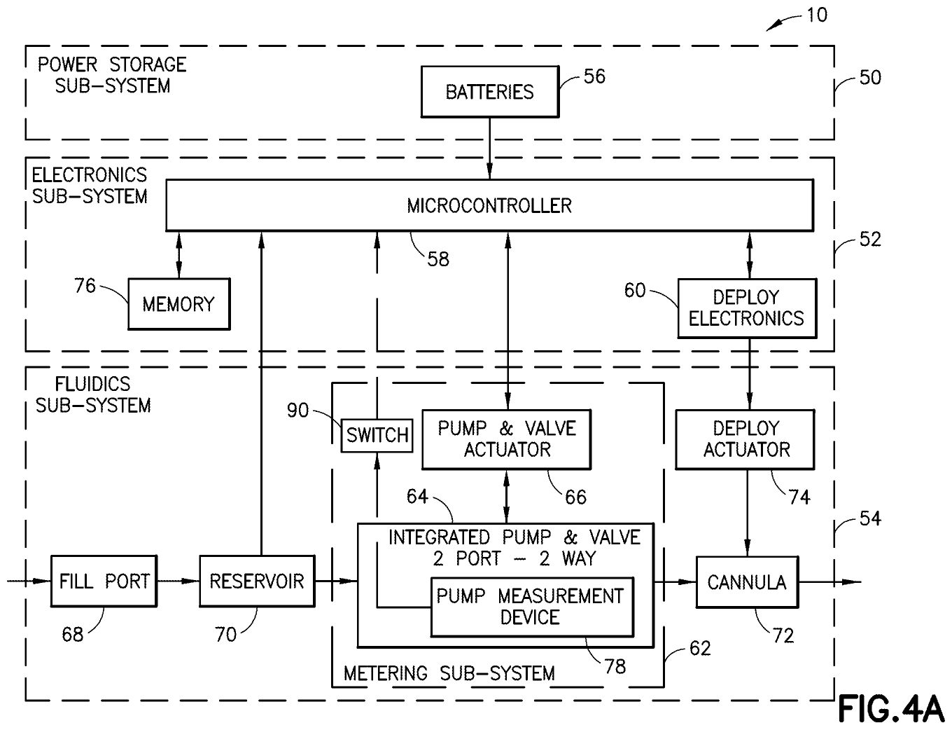

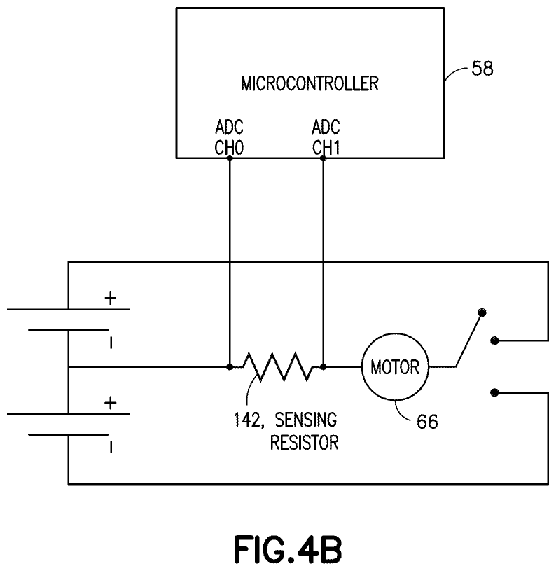

For illustrative purposes, reference is made to an example rotational metering-type pump described in commonly owned WO 2015/157174, the content of which is incorporated herein by reference in its entirety. With reference to FIGS. 1, 2, 3A, 3B and 3C, an example infusion pump (e.g., a wearable medication delivery device such as an insulin patch pump) comprises a pump assembly 20 which can be connected to a DC motor and gearbox assembly (not shown) to rotate a sleeve 24 in a pump manifold 22. A helical groove 26 is provided on the sleeve. A coupling pin 28 connected to a piston 30 translates along the helical groove to guide the retraction and insertion of the piston 30 within the sleeve 24, respectively, as the sleeve 24 rotates in one direction and then rotates in the opposite direction. The sleeve has an end plug 34. Two seals 32, 36 on the respective ends of the piston and end plug that are interior to the sleeve 24 define a cavity or chamber 38 when the piston 30 is retracted, as depicted in FIG. 3A, following an aspirate stroke and therefore ready to dispense. The volume of the chamber 38 therefore changes depending on the degree of retraction of the piston 30. The volume of the chamber 38 is negligible or essentially zero when the piston 30 is fully inserted and the seals 32, 36 are substantially in contact with each other following a dispense stroke, as depicted in FIG. 3B, and therefore ready to aspirate. Two ports 44, 46 are provided relative to the pump manifold 22, including an inlet port 44 through which medication can flow from a reservoir 70 (FIG. 4A) for the pump 64 (FIG. 4A), and an outlet port 46 through which the medication that has been drawn into the chamber 38 (e.g., by retraction of the piston 30 during an aspirate stage of operation) can be dispensed from the chamber 38 to, for example, a fluid path to a cannula 72 (FIG. 4A) in the patient by re-insertion of the piston 30 into the chamber 38.

With continued reference to FIGS. 1, 2, 3A, 3B and 3C, the sleeve 24 can be provided with an aperture (not shown) that aligns with the outlet port 46 or the inlet port 44 (i.e., depending on the degree of rotation of the sleeve 24 and therefore the degree of translation of the piston 30) to permit the medication in the chamber 38 to flow through the corresponding one of the ports 44, 46. A pump measurement device 78 (FIG. 4A) such as a sleeve rotational limit switch can be provided which has, for example, an interlock 42 and one or more detents 40 on the sleeve 24 or its end plug 34 that cooperate with the interlock 42. The interlock 42 can be mounted to the manifold 22 at each end thereof. The detent 40 at the end face of sleeve 24 is adjacent to a bump 48 of the interlock 42 when the pump 64 is in a first position whereby a side hole in the sleeve 24 is aligned with the inlet port 44 to receive fluid from the reservoir 70 into the chamber 38. Under certain conditions, such as back pressure, it is possible that friction between the piston 30 and the sleeve 24 is sufficient to cause the sleeve 24 to rotate before the piston 30 and coupling pin 28 reach either end of the helical groove 26. This could result in an incomplete volume of liquid being pumped per stroke. In order to prevent this situation, the interlock 42 prevents the sleeve 24 from rotating until the torque passes a predetermined threshold, as shown in FIG. 3A. This ensures that piston 30 fully rotates within the sleeve until the coupling pin reaches the end of the helical groove 26. Once the coupling pin 28 hits the end of the helical groove 26, further movement by the DC motor and gearbox assembly or other type of pump and valve actuator 66 (FIG. 4A) increases torque on the sleeve 24 beyond the threshold, causing the interlock 42 to flex and permit the detent 40 to pass by the bump 48. At the completion of rotation of the sleeve 24 such that its side hole is oriented with the cannula 72 or outlet port 46, the detent 40 moves past the bump 48 in the interlock 42, as shown in FIG. 3B. Another sleeve feature 41 can be provided to engage an electrical switch (e.g., an end-stop switch 90 provided on a printed circuit board 92 and disposed relative to the sleeve and/or end plug 34 to cooperate with the pump measurement device 78 as shown in FIG. 3C).

FIG. 4A is an illustrative system diagram that illustrates example components in an example medication delivery device 10 having an infusion pump such as the pump of FIGS. 1, 2, 3A, 3B and 3C. The medication delivery device 10 can include an electronics sub-system 52 for controlling operations of components in a fluidics sub-system 54 such as the pump 64 and an insertion mechanism 74 for deploying a cannula 72 for insertion into an infusion site on a patient's skin. A power storage sub-system 50 can include batteries 56, for example, for providing power to components in the electronics and fluidics sub-systems 52 and 54. The fluidics sub-system 54 can comprise, for example, an optional fill port 68 for filling a reservoir 70 (e.g., with medication), although the medication delivery device 10 can be optionally shipped from a manufacture having its reservoir already filled. The fluidics sub-system 54 also has a metering sub-system 62 comprising the pump 64 and a pump actuator 66. As described above, the pump 64 can have two ports 44, 46 and related valve sub-assembly that controls when fluid enters and leaves a pump chamber 38 via the respective ports 44, 46. One of the ports is an inlet port 44 through which fluid such as liquid medication flows from the reservoir 70 into the pump 64 as the result of a pump intake or pull stroke on a pump plunger or piston 30, for example. The other port is an outlet port 46 through which the fluid leaves the pump's chamber 38 and flows toward a cannula 72 for administration to a patient pump as the result of a pump discharge or push stroke on the pump plunger or piston 30. The pump actuator 66 can be a DC motor and gearbox assembly or other pump driving mechanism for controlling the plunger or piston 30 and other related pump parts such as a sleeve 24 that may rotate relative to the translational movement of the pump piston 30. The microcontroller 58 can be provided with an integrated or separate memory device having computer software instructions to actuate, for example, rotation of the sleeve 24 in a selected direction, translational or axial movement of a piston 30 in the sleeve 24 for an aspirate or dispense stroke, and optionally the rotation of the sleeve 24 and piston together during a valve state change as described in the above-referenced WO 2015/157174. As described below, an empty reservoir algorithm in accordance with illustrative embodiments can be provided to the microcontroller 58 to monitor pump parameter measurements and detect when an empty reservoir condition occurs relative to the infusion pump.

FIG. 4B shows an example apparatus for motor current sensing. A sensing resistor 142 is added to the PCB 92 to enable motor current measurement. The voltage drop on the sensing resistor 142 is provided into the analog-to-digital converter (ADC) of the microcontroller 58. The occlusion condition is then calculated by the microcontroller 58, and an occlusion or empty reservoir event is reported by the microcontroller 58 when, for example, a designated occlusion or empty reservoir motor current signature is detected. Other components can be used for current sensing to facilitate pump motor current measurement. For example, for a pulse width modulation (PWM) drive motor used as a pump actuator 66, motor current information can be extrapolated from PWM data.

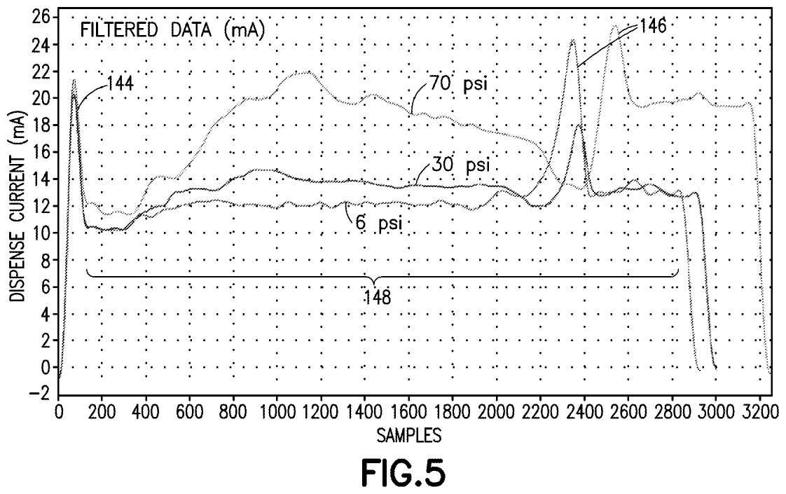

FIG. 5 depicts pump measurement data from an example delivery device indicating motor current during dispensing and variance at different pressures (e.g., 6 psi, 30 psi and 70 psi). As can be seen in FIG. 5, the pump measurement data has some consistent waveform shapes (e.g., the spikes 144 and 146 that correspond to motor start up at the beginning of a stroke and interlock operation at the end of a stroke), but also shows an increase in motor current levels as pressure increases as indicated generally at 148. Accordingly, the motor current waveform can be indicative of pressure changes from occlusion or empty reservoir condition, as stated above.

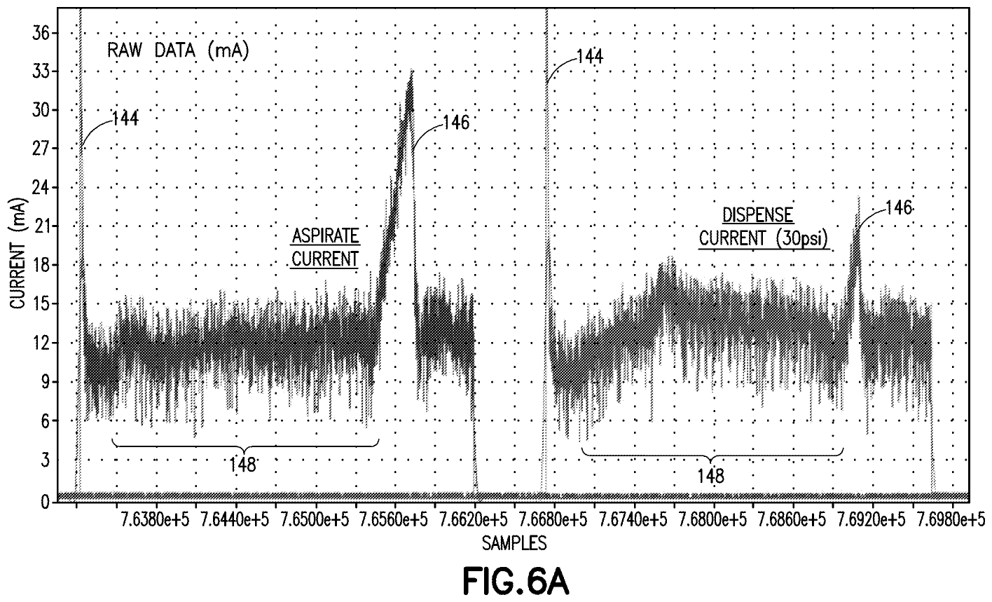

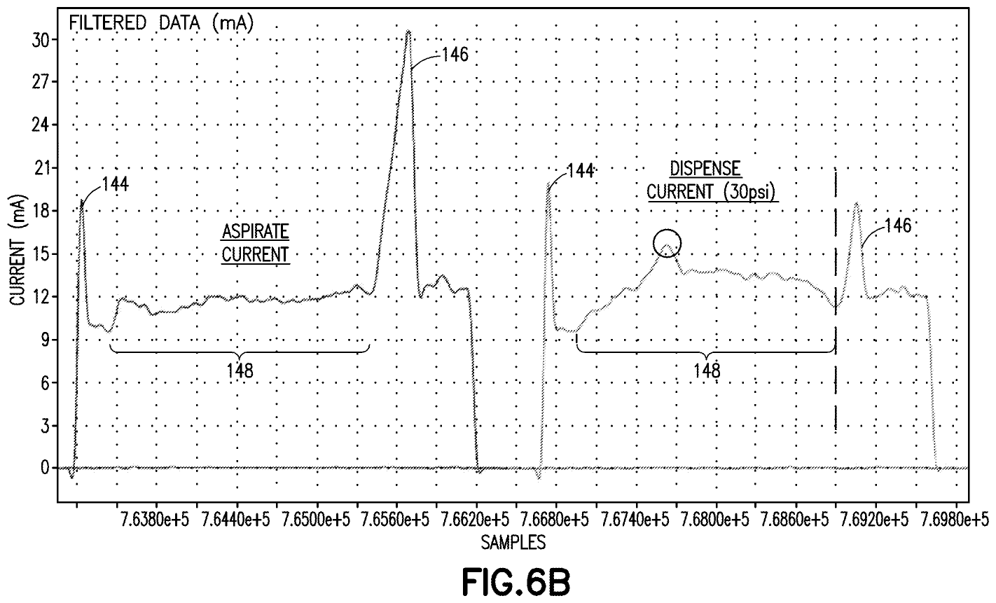

FIGS. 6A and 6B depict, respectively, raw and filtered pump measurement data (e.g., motor current) from an example delivery device during aspirate and dispense strokes. The waveform shapes or spikes 144 and 146 can be seen in both an aspirate stroke and a dispense stroke and regardless of whether the pump measurement data is raw or filtered as shown in FIGS. 6A and 6B.

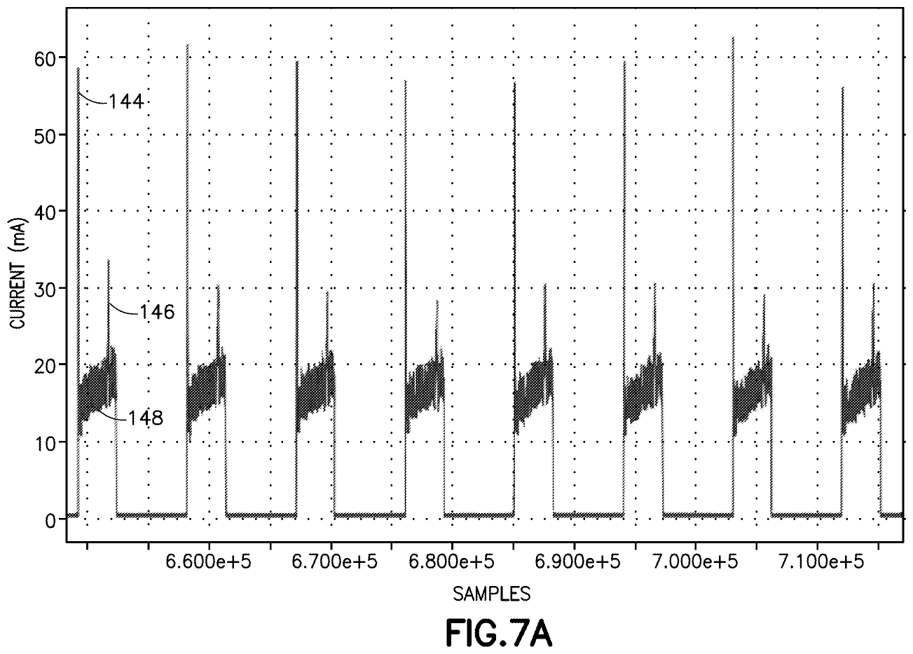

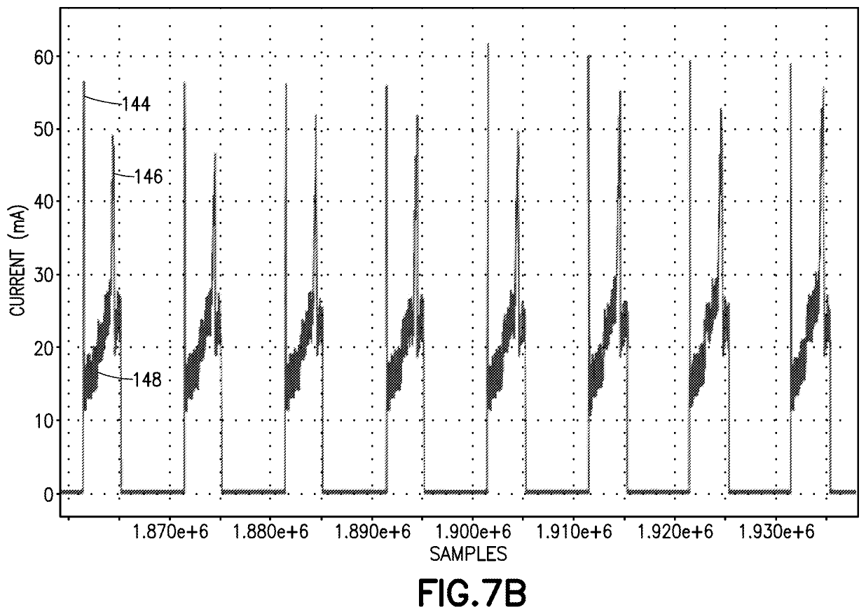

FIGS. 7A and 7B depict, respectively, changes in pump motor current during aspiration (e.g., during an aspiration stroke in the example pump shown in FIGS. 1, 2 and 3A-3C) after the pump reservoir is empty. FIG. 7A depicts filtered pump measurement data comprising measure motor current for a series of aspirate strokes when fluid remains in the reservoir 70. FIG. 7B depicts changes in the filtered pump measurement data comprising measure motor current for a series of aspirate strokes when the reservoir 70 is empty, particularly in the section 148 of the waveform between the motor current spikes 144 and 146 occurring upon start up and operation of the motor with respect to the interlock at the end of the stroke. For example, when the reservoir 70 is empty, the motor current increases to between about 22-30 mA as shown in FIG. 7B, as compared with a motor current level of about 10-21 mA when the reservoir 70 is not empty as shown in FIG. 7A. FIG. 7B also illustrates that an increase in motor current indicative of an empty reservoir condition occurs in the second half 148b of the portion 148 in the motor current waveform during an aspirate stroke.

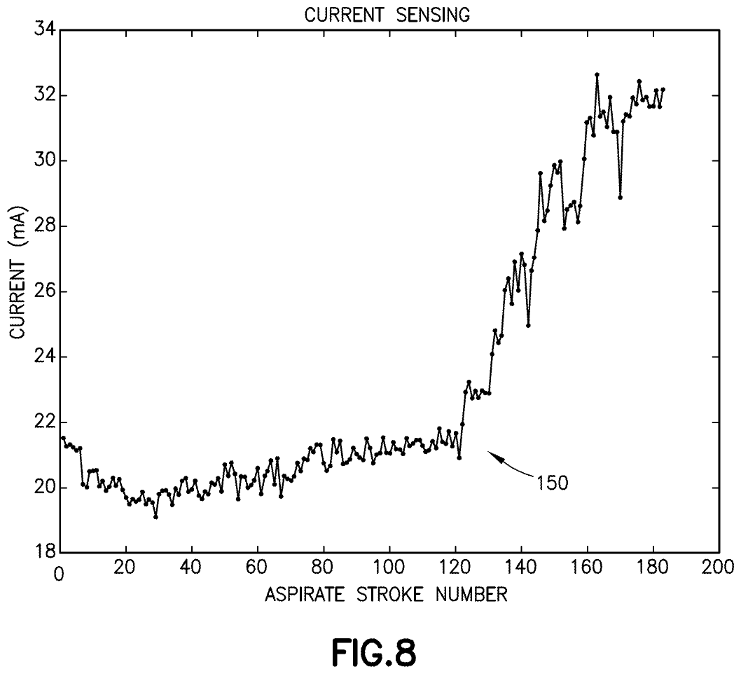

FIG. 8 depicts pump measurement data from an example delivery device (e.g., motor current) over several aspirate strokes and a point 150 within the measured motor current data that indicates an empty reservoir. Using a relatively simple averaging scheme (i.e., without using additional metrics identifying changes in the shape of a measured data waveform), FIG. 8 illustrates that an empty reservoir point 150 can be identified within the pump measurement data with relative ease and relying only on aspirate motor current.

In accordance with example embodiments of the present disclosure, criteria (e.g., thresholds and other metrics such as changes in measured data waveform shape) are established for detecting an empty reservoir condition using an empty reservoir detection algorithm provided in the control software of an infusion pump. It is to be understood that the example motor current data waveforms shown FIGS. 5, 6A, 6B, 7A, 7B and 8 are for a particular type of infusion pump, and that the pump measurement data and the criteria for detecting an empty reservoir condition can vary based on pump type, fluid type to be delivered, volume of fluid to be delivered, ambient temperature, ambient air pressure or other environmental considerations, among other factors. For example, as stated above, the pump measurement data can be, but is not limited to any of motor current, motor voltage, motor drive time, motor coast time, delivery pulse energy, motor drive count, motor coast count, and delta encoder count, among other pump operating parameters. In addition, the measured parameter can be a feature of these signals or a combination of features from different signals. The criteria for detecting an empty reservoir condition can be determined empirically, for example, by testing a particular infusion pump, reservoir and fluid set up, identifying patterns in the measured pump parameter test data, and designating the criteria based thereon for use by the empty reservoir detection algorithm.

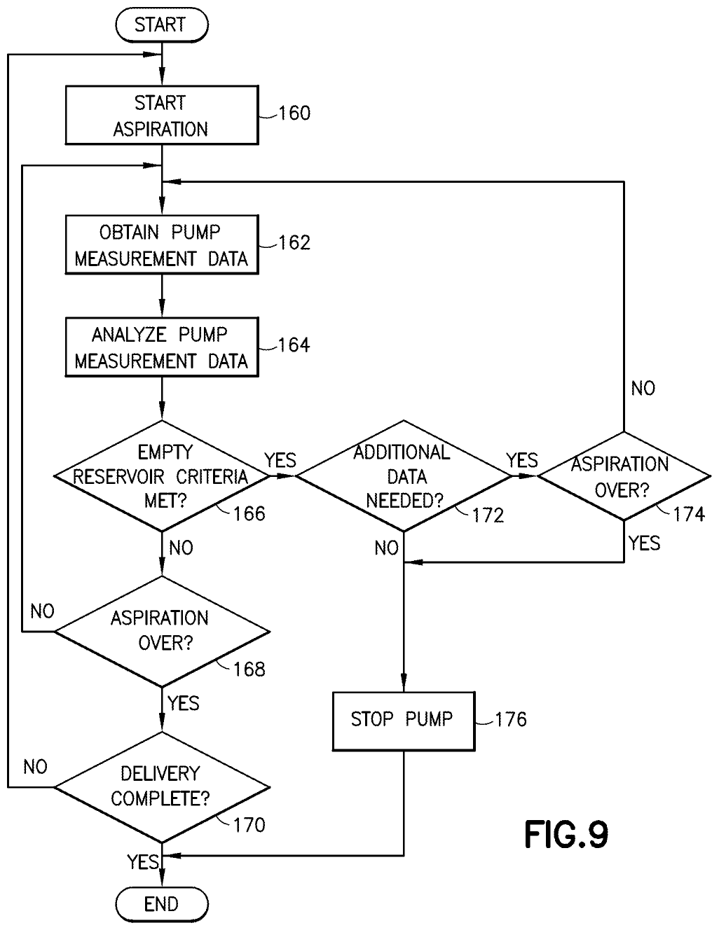

FIG. 9 is a flow chart of illustrative operations of an example medication delivery device performing an empty reservoir detection algorithm in accordance with an illustrative embodiment of the present invention. A microcontroller 58 or other processing device for controlling pump operation starts aspiration (e.g., an aspirate stroke) in accordance with the empty reservoir detection algorithm (block 160) and starts obtaining pump measurement data during the aspiration (block 162). The microcontroller 58 analyzes the pump measurement data (block 164) to determine if designated empty reservoir criteria are satisfied (block 166). For example, the criteria can be empirically determined using testing of a given pump and fluid and reservoir size and fluid delivery amounts. The criteria can comprise selected thresholds or ranges of values of measured pump data that indicate elevated pressure(s) associated with an empty reservoir. For example, multiple measured data points can be averaged and then the averaged pump measurement value, or respective samples of data point values, within a given period of the aspiration can be compared to a threshold T_{EMPTY}. The analysis in block 164 can also include waveform shape or area under curve analysis of a selected portion(s) of the pump measurement data waveform obtained during the aspiration to determine if other criteria (e.g., shape-related metrics such as slope, area under curve, and the like) are satisfied and indicate an empty reservoir condition. For example, as stated above, the microcontroller 58 can be programmed to look at pump measurement data in the second half of an aspirate stroke the exceeds a threshold T_{EMPTY} of 22 mA or higher. The microcontroller 58 can also take into consideration the motor runtime, or number of aspirate strokes, or estimated fluid amount delivered, to analyze some aspirate strokes differently than others for determining if empty reservoir condition criteria are satisfied. For example, the microcontroller 58 can be configured to disregard one or more of the pump measurements obtained during one or more portions of the duration of the aspirate operation characterized by transient increases therein from normal operation of the pumping mechanism.

With continued reference to FIG. 9, if the empty reservoir condition criteria are not met (block 166) and the aspiration is not yet over (block 168), then the microcontroller 58 is programmed to continue obtaining pump measurement data (block 162) and analyzing it (block 164). If, however, the aspiration is complete (block 168) and the intended delivery of fluid is complete (block 170), then the medication delivery device can be either turned off and/or replaced, depending on the type of pump and reservoir it uses. Alternatively, for a metered reciprocating pump with alternating aspirate and dispense strokes to deliver an incremental amount of fluid in a metered chamber 38, the completion of delivery (block 170) can refer to delivery of the incremental fluid amount in the metered chamber 38, and the microcontroller can wait until another dispense stroke occurs before starting another aspirate stroke (block 160).

With continued reference to FIG. 9, if empty reservoir condition criteria are met (block 166), additional pump measurement data (block 172) may need to be obtained (block 162) and analyzed (block 164) for other thresholds or waveform shape-related metrics and/or for other portions of the aspiration before an empty or end of life (EOL) determination for the reservoir can be made. If no additional pump measurement data is needed, then the microcontroller 58 can be configured by the empty reservoir detection algorithm to stop the pump (block 176), thereby avoiding dose inaccuracies from over-pumping. On the other hand, if the aspiration is not yet complete and additional pump measurement data is needed, then the microcontroller 58 is programmed to continue obtaining pump measurement data (block 162) and analyzing it (block 164).

The same things can potentially be calculated based on other measurable characteristics of the electrical system. For example, motor voltage can be measured instead of, or in addition to, motor current, and is characterized by a similar shape change to that shown in FIG. 7B when the reservoir is empty because the motor draws more current by slowing down, which decreases back EMF and impedance of the motor. With decreased impedance, current increases through the motor. At the same time, the voltage source has some internal resistance (impedance). Because the impedance across the motor is changing and is on a similar scale as the battery impedance, the voltage drop seen across the battery changes too.

As stated above, a typical solution for empty reservoir detection is to place an additional sensor in the pump control system and report empty reservoir status when detected by the sensor. Adding a sensor, however, has the drawbacks of increasing the complexity of the system (e.g., mechanical, electrical, and/or software complexity), increasing system power consumption, and/or increasing pump cost. These drawbacks can be particularly disadvantageous to a wearable pump design wherein all or part of the pump is intended to be disposable once the reservoir 70 is emptied or the pump 64 has been used a selected amount of time and/or used to deliver a selected amount of medication.

In accordance with illustrative embodiments, empty reservoir detection is accomplished without an additional component. Instead a microcontroller 58 or other processing device for controlling pump operation can be further controlled to determine when a motor parameter measurement(s) is/are outside a designated range of normal operating conditions and therefore indicate(s) an empty reservoir (e.g., satisfies a given threshold or other metric(s)), and terminates pump operation, and optionally generates an indication of empty reservoir status. The pump reservoir 70 and/or the entire medication delivery device 10 can, in turn, be replaced, thereby ensuring that the patient is receiving the full intended dosage that is provided under normal operating conditions.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. An infusion device comprises:
a pump comprising a chamber of fluid, and a pumping mechanism configured to control aspiration of a volume of fluid from a reservoir into the chamber during an aspirate operation and dispensing the fluid from the chamber during a dispense operation; and
a processing device configured to analyze one or more pump measurements obtained during the aspirate operation and determine when the one or more of the pump measurements satisfies a designated metric related to an empty reservoir condition of the reservoir.

2. An infusion device as claimed in claim 1, wherein the pump measurements are measurements of motor current of the pump.

3. An infusion device as claimed in claim 1, wherein the processing device is configured to terminate operation of the pumping mechanism when the one or more of the pump measurements satisfies the designated metric.

4. An infusion device as claimed in claim 1, wherein the processing device is configured to analyze additional pump measurements when the one or more of the pump measurements satisfies the designated metric, and determine when the additional pump measurements satisfies the designated metric before terminate operation of the pumping mechanism.

5. An infusion device as claimed in claim 4, wherein the processing device is configured to terminate operation of the pumping mechanism when the additional pump measurements satisfy the designated metric.

6. An infusion device as claimed in claim 1, wherein the designated metric is one or more metrics chosen from a pressure threshold corresponding to a pump measurement value exceeded when the reservoir is empty, a range of pump measurement values indicating a pressure above normal operating pressure of the pump, and a designated shape of a signal waveform corresponding to the pump measurements indicating a pressure above normal operating pressure of the pump.

7. An infusion device as claimed in claim 1, wherein the processing device is configured to analyze one or more of the pump measurements obtained during a selected portion of the duration of the aspirate operation.

8. An infusion device as claimed in claim 1, wherein the processing device is configured to disregard one or more of the pump measurements obtained during one or more portions of the duration of the aspirate operation characterized by transient increases therein from normal operation of the pumping mechanism.

9. An infusion device as claimed in claim 1, wherein the pump measurements are chosen from one or more of pump motor current, pump motor voltage, pump encoder count, pump motor drive count, and pump motor drive time.
